(19) United States
(12) Patent Application Publication
(10) Pub. No.: US 2017/0105705 A1
(43) Pub. Date: Apr. 20, 2017

(54) ADAPTIVE DEMODULATION METHOD AND APPARATUS FOR ULTRASOUND IMAGE

(71) Applicant: SAMSUNG ELECTRONICS CO., LTD., Suwon-si (KR)

(72) Inventors: Hee-chul YOON, Seoul (KR); Kang-won JEON, Yongin-si (KR); Yong-sup PARK, Seoul (KR); Hyun-taek LEE, Seoul (KR)

(73) Assignee: SAMSUNG ELECTRONICS CO., LTD., Suwon-si (KR)

(21) Appl. No.: 15/129,156

(22) PCT Filed: Mar. 17, 2015
(86) PCT No.: PCT/KR2015/002549

§ 371 Date: Sep. 26, 2016

(30) Foreign Application Priority Data
Mar. 26, 2014 (KR) 10-2014-0035380

(51) Int. Cl. A61B 8/08; A61B 5/00; A61B 8/14; G01S 7/52

(52) U.S. Cl. CPC A61B 8/5269; G01S 7/52085; A61B 5/7228; A61B 5/7246; A61B 5/7221; A61B 8/5207; A61B 8/14

(57) ABSTRACT

Disclosed are a method and an apparatus, which enhance a quality of an ultrasound image to provide an improved image. An adaptive demodulation method includes acquiring input radio frequency (RF) data, quadrature-demodulating the input RF data to output an inphase-quadrature (IQ) signal, determining a valid region for the input RF data, and estimating attenuation of a frequency of the IQ signal, based on data included in the valid region among the input RF data and performing frequency compensation corresponding to the estimated attenuation of the frequency.

### TECHNICAL FIELD

One or more exemplary embodiments relate to an adaptive demodulation method and apparatus, and more particularly, to an adaptive demodulation method and apparatus in which performance is enhanced by accurately estimating a frequency.

### BACKGROUND ART

Various imaging apparatuses are used for noninvasively observing the inside of a human body. In particular, ultrasound images have more stability than other images using X-rays, and since a human body is not exposed to radiation, the ultrasound images are stable. Therefore, ultrasound images are widely used. Ultrasound diagnosis apparatuses transmit ultrasound signals generated by transducers of a probe to an object and receive echo signals reflected from the object, thereby obtaining at least one image of an internal part of the object. In particular, ultrasound diagnosis apparatuses are used for medical purposes including observation of the interior of an object, detection of foreign substances, and diagnosis of damage to the object.

A physical characteristic (for example, attenuation) of an ultrasound wave is greatly changed depending on objects (for example, human bodies of patients). For this reason, a quality of an ultrasound image is degraded depending on a feature of an object. Therefore, a method of providing a high-quality image irrespective of a feature of an object is needed.

### DISCLOSURE

### Technical Problem

One or more exemplary embodiments include a method and an apparatus, which enhance a quality of an ultrasound image to provide an improved image.

### Technical Solution

According to one or more exemplary embodiments, an adaptive demodulation method includes: a) acquiring input radio frequency (RF) data; b) quadrature-demodulating the input RF data to output an inphase-quadrature (IQ) signal; c) determining a valid region for the input RF data; and d) estimating attenuation of a frequency of the IQ signal, based on data included in the valid region from among the input RF data and performing frequency compensation corresponding to the estimated attenuation of the frequency.

### Advantageous Effects

One or more exemplary embodiments include an image demodulation method and apparatus in which a performance of restoring an image is enhanced by detecting a region where attenuation of a frequency is validly estimated.

### DESCRIPTION OF DRAWINGS

These and/or other aspects will become apparent and more readily appreciated from the following description of the exemplary embodiments, taken in conjunction with the accompanying drawings in which:

FIG. 1 is a flowchart illustrating a process of demodulating an image, according to an exemplary embodiment;

FIG. 2 is a block diagram illustrating an image demodulation apparatus according to an exemplary embodiment;

FIG. 3 is a conceptual diagram illustrating a valid region according to an exemplary embodiment;

FIG. 4 is a structure diagram illustrating an image demodulation apparatus according to an exemplary embodiment;

FIG. 5 is a structure diagram illustrating an image demodulation apparatus according to another exemplary embodiment;

FIG. 6 is a structure diagram illustrating an image demodulation apparatus according to another exemplary embodiment;

FIG. 7 is a graph showing a result obtained by estimating a center frequency; and

FIG. 8 is a block diagram illustrating an ultrasound diagnosis according to an exemplary embodiment.

### BEST MODE

Additional aspects will be set forth in part in the description which follows and, in part, will be apparent from the description, or may be learned by practice of the presented exemplary embodiments.

According to one or more exemplary embodiments, an adaptive demodulation method includes: a) acquiring input radio frequency (RF) data; b) quadrature-demodulating the input RF data to output an inphase-quadrature (IQ) signal; c) determining a valid region for the input RF data; and d) estimating attenuation of a frequency of the IQ signal, based on data included in the valid region from among the input RF data and performing frequency compensation corresponding to the estimated attenuation of the frequency.

Step c) may include: acquiring a cross-correlation of the input RF data; and determining the valid region, based on the cross-correlation.

The acquiring of the cross-correlation may include acquiring a cross-correlation between previous beamformed data and current beamformed data.

Step c) may further include performing beamforming of a virtual scan line by using ith scan line channel data and i+1st scan line channel data, and the acquiring of the cross-correlation may include acquiring a cross-correlation of beamformed data.

The determining of the valid region may include: acquiring a signal-to-noise (SNR) value from previous beamformed data and current beamformed data; and determining the valid region, based on the SNR value.

Step d) may include: acquiring an auto-correlation of the IQ signal; performing polynomial fitting, based on the auto-correlation and the valid region; and performing frequency shift compensation, based on a result of the polynomial fitting.

According to one or more exemplary embodiments, an adaptive demodulation apparatus includes: an input data acquirer that acquires input radio frequency (RF) data; a quadrature demodulator that quadrature-demodulates the input RF data to output an inphase-quadrature (IQ) signal; a valid region determiner that determines a valid region for the input RF data; and a frequency compensator that estimates attenuation of a frequency of the IQ signal, based on data included in the valid region from among the input RF data and performs frequency compensation corresponding to the estimated attenuation of the frequency.

The valid region determiner may include: a cross-correlator that acquires a cross-correlation of the input RF data; a polynomial function fitting unit that performs polynomial fitting on the input RF data, based on the cross-correlation; and a valid region selector that determines the valid region, based on a result of the polynomial fitting.

The cross-correlator may acquire a cross-correlation between previous beamformed data and current beamformed data.

The valid region determiner may include: a beamformer that performs beamforming of a virtual scan line by using ith scan line channel data and i+1st scan line channel data; a cross-correlator that acquires a cross-correlation, based on the virtual scan line; a polynomial function fitting unit that performs polynomial fitting, based on the cross-correlation; and a valid region selector that determines the valid region, based on a result of the polynomial fitting by the polynomial function fitting unit.

The valid region determiner may include: a signal-to-noise (SNR) estimator that estimates an SNR value from previous beamformed data and current beamformed data; a polynomial function fitting unit that performs polynomial fitting, based on the estimated SNR value; and a valid region selector that determines the valid region, based on a result of the polynomial fitting by the polynomial function fitting unit.

The frequency compensator may include: an auto-correlator that acquires an auto-correlation of the IQ signal; a polynomial function fitting unit that performs polynomial fitting, based on the auto-correlation and the valid region; and a frequency shift compensator that performs frequency shift compensation, based on a result of the polynomial fitting.

A non-transitory computer-readable storage medium according to exemplary embodiments may store a program for executing the adaptive demodulation method.

### MODE FOR INVENTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description.

Exemplary embodiments of the inventive concept capable of being easily embodied by one of ordinary skill in the art will now be described in detail with reference to the accompanying drawings. In this regard, the present embodiments may have different forms and should not be construed as being limited to the descriptions set forth herein. In the accompanying drawings, a portion irrelevant to a description of the inventive concept will be omitted for clarity. Moreover, like reference numerals refer to like elements throughout.

Terms used in the inventive concept have been selected as general terms which are widely used at present, in consideration of the functions of the inventive concept, but may be altered according to the intent of an operator of ordinary skill in the art, conventional practice, or introduction of new technology. Also, if there is a term which is arbitrarily selected by the applicant in a specific case, in which case a meaning of the term will be described in detail in a corresponding description portion of the inventive concept. Therefore, the terms should be defined on the basis of the entire content of this specification instead of a simple name of each of the terms.

In this disclosure below, when one part (or element, device, etc.) is referred to as being ‘connected’ to another part (or element, device, etc.), it should be understood that the former may be ‘directly connected’ to the latter, or ‘electrically connected’ to the latter via an intervening part (or element, device, etc.). When a part “includes” or “comprises” an element, unless there is a particular description contrary thereto, the part can further include other elements, not excluding the other elements.

Throughout the specification, it will also be understood that when a component “includes” an element, unless there is another opposite description thereto, it should be understood that the component does not exclude another element and may further include another element. In addition, terms such as “. . . unit”, “. . . module”, or the like refer to units that perform at least one function or operation, and the units may be implemented as hardware or software or as a combination of hardware and software.

Throughout the specification, an “ultrasound image” refers to an image of an object, which is obtained using ultrasound waves. Furthermore, an “object” may be a human, an animal, or a part of a human or animal. For example, the object may be an organ (e.g., the liver, the heart, the womb, the brain, a breast, or the abdomen), a blood vessel, or a combination thereof. Also, the object may be a phantom. The phantom means a material having a density, an effective atomic number, and a volume that are approximately the same as those of an organism.

Throughout the specification, a “user” may be, but is not limited to, a medical expert, for example, a medical doctor, a nurse, a medical laboratory technologist, or a medical imaging expert, or a technician who repairs medical apparatuses.

In this disclosure below, a valid region denotes a region in which frequency estimation by an image demodulation apparatus is valid. Also, in an ultrasound wave, as a depth of an object becomes deeper, a frequency is attenuated. Frequency estimation denotes estimating an attenuation of a frequency for compensating for an attenuated frequency.

Hereinafter, exemplary embodiments will be described in detail with reference to the accompanying drawings.

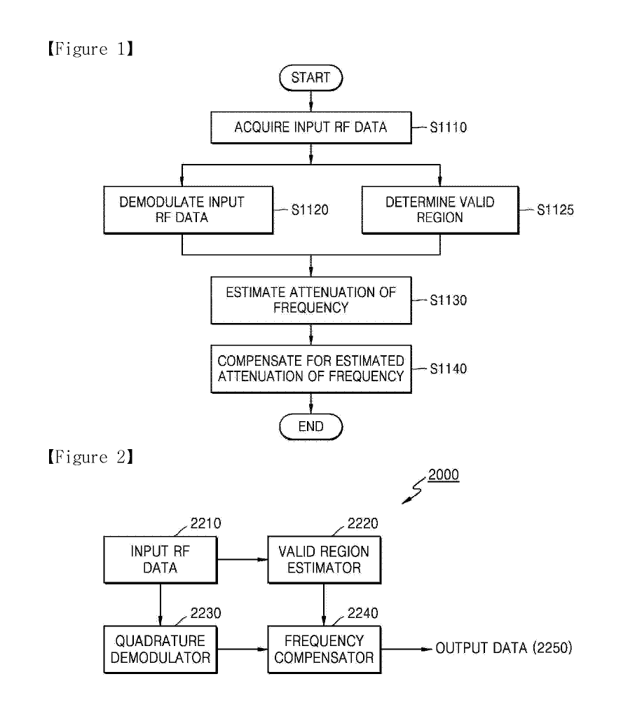

FIG. 1 is a flowchart illustrating a process of demodulating an image, according to an exemplary embodiment.

In operation S1110, an image demodulation apparatus may acquire input radio frequency (RF) data. Here, the input RF data may be data which is acquired based on an echo signal of an ultrasound signal transmitted from an ultrasound diagnostic apparatus.

Subsequently, in operation S1120, the image demodulation apparatus may demodulate the input RF data. Here, the image demodulation apparatus may quadrature-demodulate the input RF data. The image demodulation apparatus may output an inphase-quadrature (IQ) signal as a result obtained by demodulating the input RF data.

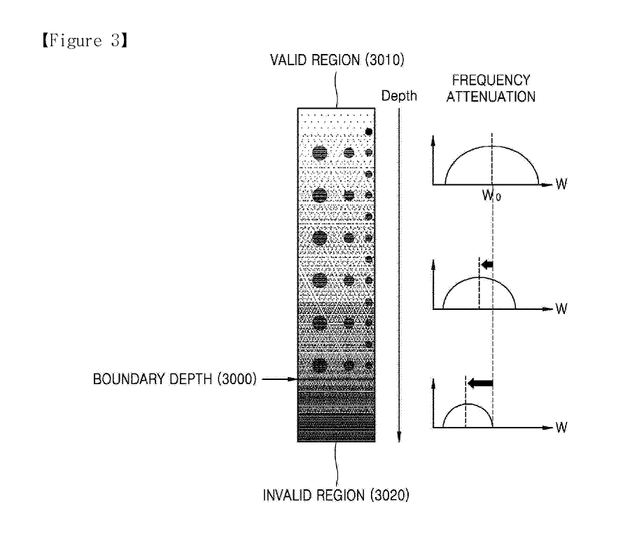

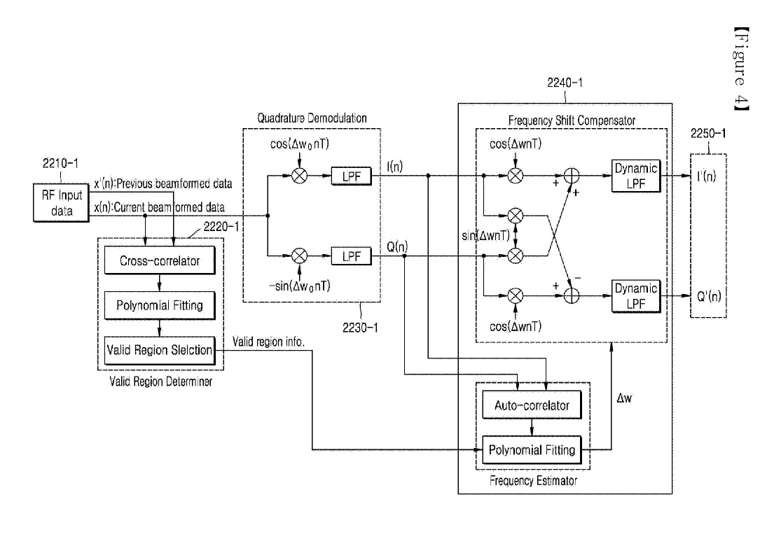

As illustrated in FIG. 3, in the acquired input RF data, as a depth becomes deeper, a frequency is attenuated. A depth denotes a distance from a surface of an object to the inside. In operation S1130, the image demodulation apparatus may estimate a center frequency for compensating for attenuation caused by a depth. However, when a depth is equal to or greater than a boundary depth (3000), the center frequency estimated by the image demodulation apparatus becomes higher as the depth increases. That is, when a depth of an image is outside a valid region (3010) and is equal to or greater than the boundary depth (3000), an efficiency of frequency estimation is reduced by data included in an invalid region (3020).

Therefore, in operation S1125, the image demodulation apparatus may determine a valid region for the input RF data. A method of determining the valid region may be variously implemented.

According to an exemplary embodiment, in operation S1125, the image demodulation apparatus may acquire a cross-correlation of the input RF data. Here, a cross-correlation value may be a cross-correlation value between previous beamformed data and current beamformed data. The image demodulation apparatus may determine a valid region, based on the cross-correlation. That is, the image demodulation apparatus may determine, as the valid region, a region in which the cross-correlation value is equal to or greater than a threshold value. The threshold value may be set by using an experimental statistic value.

Moreover, according to another exemplary embodiment, in operation S1125, the image demodulation apparatus may perform beamforming of a virtual scan line by using ith scan line channel data and i+1st scan line channel data. The image demodulation apparatus may acquire a cross-correlation of the beamformed virtual scan line. The image demodulation apparatus may determine a valid region, based on the cross-correlation. That is, the image demodulation apparatus may determine, as the valid region, a region in which a value of the cross-correlation is equal to or greater than a threshold value. The threshold value may be set by using an experimental statistic value.

Moreover, according to another exemplary embodiment, in operation S1125, the image demodulation apparatus may acquire a signal-to-noise ratio (SNR) value from previous beamformed data and current beamformed data. The image demodulation apparatus may determine, as a valid region, a region in which an SRN is equal to or greater than a threshold value. The threshold value may be set by using an experimental statistic value.

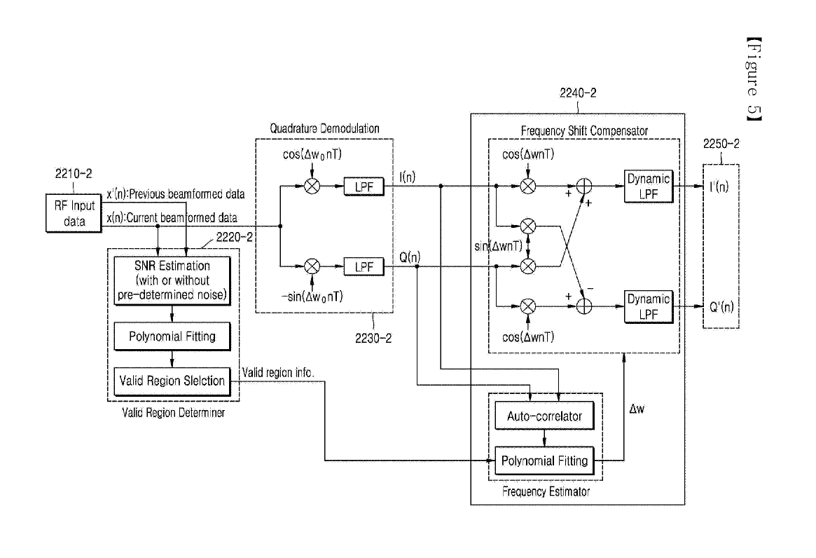

A detailed exemplary embodiment of determining a valid region will be described in detail with reference to FIGS. 4 to 6.

The image demodulation apparatus may perform frequency estimation for the valid region which is determined in operation S1130. That is, the image demodulation apparatus may perform an auto-correlation from the IQ signal and perform polynomial fitting, based on the determined valid region and a result obtained by performing the auto-correlation.

Subsequently, in operation S1135, the image demodulation apparatus may compensate for attenuation of a frequency which is estimated in operation S1130. That is, the image demodulation apparatus may perform frequency shift compensation on the IQ signal, based on the frequency which is estimated in operation S1130.

FIG. 2 is a block diagram illustrating an image demodulation apparatus 2000 according to an exemplary embodiment.

The image demodulation apparatus 2000 according to an exemplary embodiment may include a valid region estimator 2220 that estimates a valid region, a quadrature demodulator 2230 that demodulates input RF data 2210, and a frequency compensator 2240 that estimates attenuation of a frequency of an IQ signal and performs frequency compensation corresponding to the estimated attenuation of the frequency.

The quadrature demodulator 2230 may demodulate the input RF data 2210. Here, the quadrature demodulator 2230 may quadrature-demodulate the input RF data 2210. The quadrature demodulator 2230 may output the IQ signal as a result obtained by demodulating the input RF data 2210.

As illustrated in FIG. 3, in the acquired input RF data, as a depth becomes deeper, a frequency is attenuated. A depth denotes a distance from a surface of an object to the inside. The frequency compensator 2240 may estimate a center frequency for compensating for attenuation caused by a depth. When a depth is equal to or greater than the boundary depth (3000), the center frequency estimated by the image demodulation apparatus becomes higher as the depth increases. That is, when a depth of an image is out of the valid region (3010) and is equal to or greater than the boundary depth (3000), an efficiency of frequency estimation is reduced by data included in the invalid region (3020).

Therefore, the valid region estimator 2220 may determine a valid region for the input RF data 2210. A method of determining the valid region may be variously implemented.

According to an exemplary embodiment, the valid region estimator 2220 may acquire a cross-correlation of the input RF data. Here, the cross-correlation may be a cross-correlation between previous beamformed data and current beamformed data. The image demodulation apparatus may determine a valid region, based on the cross-correlation. That is, the valid region estimator 2220 may determine, as the valid region, a region in which the cross-correlation value is equal to or greater than a threshold value. The threshold value may be set by using an experimental statistic value.

Moreover, according to another exemplary embodiment, the valid region estimator 2220 may perform beamforming of the virtual scan line by using the ith scan line channel data and the i+1st scan line channel data. The valid region estimator 2220 may acquire a cross-correlation of the beamformed virtual scan line. The valid region estimator 2220 may determine a valid region, based on the cross-correlation. That is, the valid region estimator 2220 may determine, as the valid region, a region in which a value of the cross-correlation is equal to or greater than a threshold value. The threshold value may be set by using an experimental statistic value.

Moreover, according to another exemplary embodiment, the valid region estimator 2220 may acquire an SNR value from previous beamformed data and current beamformed data. The valid region estimator 2220 may determine, as a valid region, a region in which an SRN is equal to or greater than a threshold value. The threshold value may be set by using an experimental statistic value.

A detailed exemplary embodiment of determining a valid region will be described in detail with reference to FIGS. 4 to 6.

The frequency compensator 2240 may include a frequency estimator (not shown) and a frequency shift compensator (not shown). The frequency estimator (not shown) may perform frequency estimation for the valid region determined by the valid region estimator 2220. The frequency estimator (not shown) may include an auto-correlator (not shown) and a polynomial function fitting unit (not shown). The auto-correlator (not shown) may acquire an auto-correlation, based on the IQ signal output from the quadrature demodulator 2230. The polynomial function fitting unit (not shown) may perform polynomial fitting, based on the auto-correlation and the valid region. The frequency shift compensator (not shown) may perform frequency shift compensation, based on a result of the polynomial fitting. The frequency compensator may perform frequency compensation on the IQ signal to output data 2250.

FIG. 3 is a conceptual diagram illustrating a valid region according to an exemplary embodiment.

As illustrated in FIG. 3, an original signal may have a frequency w0. However, as a depth increases, a frequency may be more lowered than w0. The image demodulation apparatus may validly estimate a frequency which is attenuated in the valid region (3010) having a depth equal to or less than the boundary depth 3000. However, it is difficult for the image demodulation apparatus to perform accurate frequency estimation for the invalid region 3020 having a depth greater than the boundary depth 3000.

FIG. 4 is a structure diagram illustrating an image demodulation apparatus according to an exemplary embodiment.

The image demodulation apparatus according to an exemplary embodiment may include a valid region determiner 2220-1, a quadrature demodulator 2230-1, and a frequency compensator 2240-1.

The image demodulation apparatus may acquire input RF data 2210-1. The quadrature demodulator 2230-1 may quadrature-demodulate current beamformed data x(n) to output an IQ signal.

Moreover, the valid region determiner 2220-1 may include a cross-correlator, a polynomial function fitting unit, and a valid region selector. The cross-correlator may acquire a cross-correlation between previous beamformed data and current beamformed data. The polynomial function fitting unit may perform polynomial fitting on the input RF data 2210-1, based on the cross-correlation. Subsequently, the valid region selector may determine a valid region, based on the cross-correlation. For example, the valid region selector may select, as the valid region, a region in which a value of the cross-correlation is equal to or greater than a threshold value. The threshold value may be set by using an experimental statistic value. The valid region determiner 2220-1 may supply information about the determined valid region to the frequency compensator 2240-1.

The frequency compensator 2240-1 may include a frequency estimator and a frequency shift compensator. The frequency estimator may include an auto-correlator, which acquires an auto-correlation, and a polynomial function fitting unit that performs polynomial fitting. The polynomial function fitting unit may estimate a frequency, based on the auto-correlation and valid region information. The frequency estimator may supply Δw to the frequency shift compensator, based on the estimated frequency.

The frequency shift compensator may correct a frequency of an IQ signal, based on Δw and pass the corrected frequency through a low-pass filter (LPF), thereby outputting output data 2250-1 of which a frequency is compensated for based on the valid region.

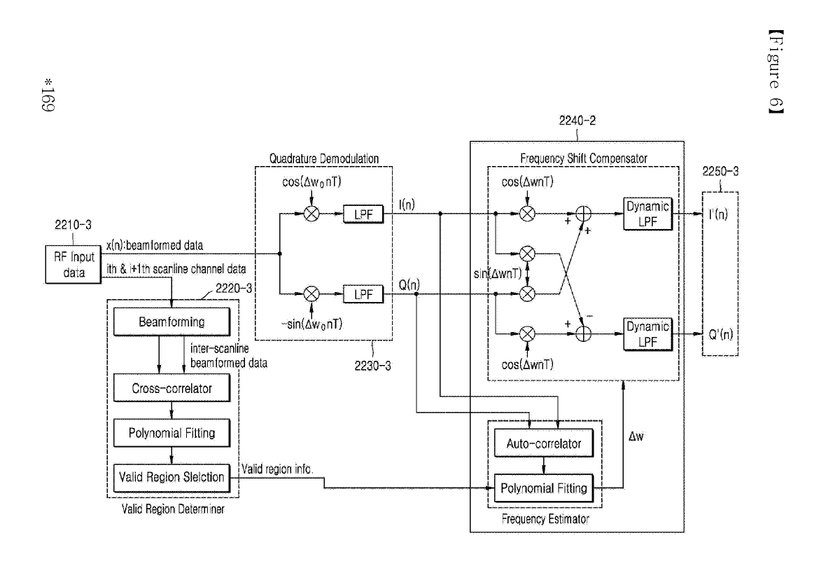

FIG. 5 is a structure diagram illustrating an image demodulation apparatus according to another exemplary embodiment.

The image demodulation apparatus according to another exemplary embodiment may include a valid region determiner 2220-2, a quadrature demodulator 2230-2, and a frequency compensator 2240-2.

The image demodulation apparatus may acquire input RF data 2210-2. The quadrature demodulator 2230-2 may quadrature-demodulate current beamformed data x(n) to output an IQ signal.

Moreover, the valid region determiner 2220-2 may include an SNR estimator that estimates an SNR, a polynomial function fitting unit, and a valid region selector. The SNR estimator may estimate an SNR by using a previously estimated noise value or a noise value estimated from a current image. For example, the SNR estimator may acquire an SNR value from previous beamformed data and current beamformed data.

The polynomial function fitting unit may perform polynomial fitting, based on the SNR value. Subsequently, the valid region selector may determine a valid region, based on the SNR value. For example, the valid region selector may select, as the valid region, a region in which an SNR is equal to or greater than a threshold value. The threshold value may be set by using an experimental statistic value. The valid region determiner 2220-2 may supply information about the determined valid region to the frequency compensator 2240-2.

The frequency compensator 2240-2 may include a frequency estimator and a frequency shift compensator. The frequency estimator may include an auto-correlator, which acquires an auto-correlation, and a polynomial function fitting unit that performs polynomial fitting. The polynomial function fitting unit may estimate a frequency, based on the auto-correlation and valid region information. The frequency estimator may supply Δw to the frequency shift compensator, based on the estimated frequency.

The frequency shift compensator may correct a frequency of an IQ signal, based on Δw and pass the corrected frequency through a low-pass filter (LPF), thereby outputting output data 2250-2 of which a frequency is compensated for based on the valid region.

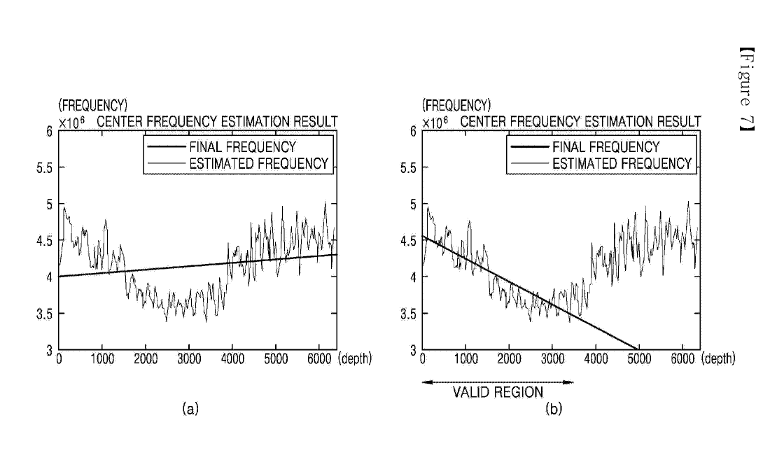

FIG. 6 is a structure diagram illustrating an image demodulation apparatus according to another exemplary embodiment.

The image demodulation apparatus according to another exemplary embodiment may include a valid region determiner 2220-3, a quadrature demodulator 2230-3, and a frequency compensator 2240-3.

The image demodulation apparatus may acquire input RF data 2210-3. The quadrature demodulator 2230-3 may quadrature-demodulate current beamformed data x(n) to output an IQ signal.

Moreover, the valid region determiner 2220-3 may include a beamformer, a cross-correlator, a polynomial function fitting unit, and a valid region selector. The beamformer may perform beamforming of two virtual scan lines by using ith scan line channel data and i+1st scan line channel data. The cross-correlator may acquire a cross-correlation, based on beamformed data.

FIG. 7 is a graph showing a result obtained by estimating a center frequency.

Referring to FIG. 7, as a depth decreases, an estimated frequency is reduced. However, when a depth is greater than about 3,500, it is estimated that a frequency increases. Therefore, by using a result obtained by estimating a frequency for each depth, as shown in FIG. 7 (a), a finally acquired frequency does not match attenuation of an actual frequency.

However, according to an exemplary embodiment, a region in which a depth is about 0 to about 3,500 may be determined as a valid region. By using only a result of frequency estimation for the valid region, a finally acquired frequency is more similar to attenuation of an actual frequency.

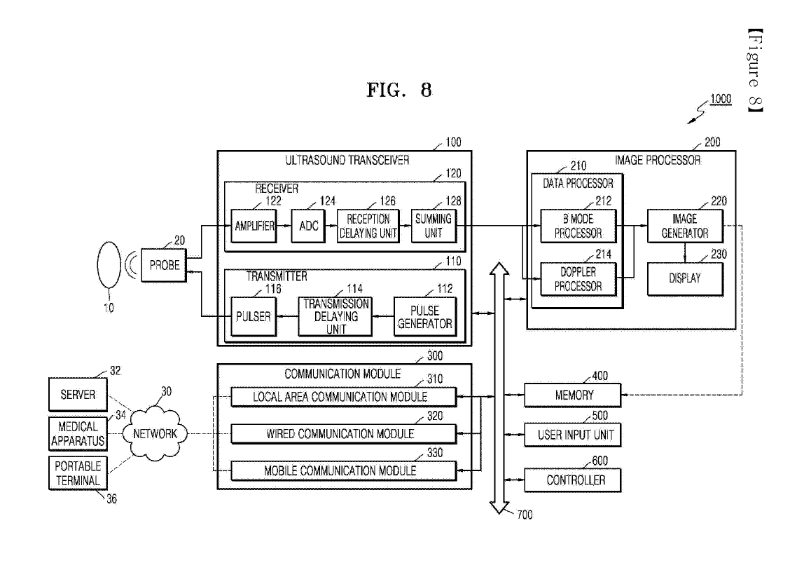

FIG. 8 is a block diagram showing an ultrasound diagnosis apparatus 1000 according to an embodiment. Referring to FIG. 1, the ultrasound diagnosis apparatus 1000 may include a probe 20, an ultrasound transceiver 100, an image processor 200, a communication module 300, a display 300, a memory 400, an input device 500, and a controller 600, which may be connected to one another via buses 700.

The ultrasound diagnosis apparatus 1000 may be a cart type apparatus or a portable type apparatus. Examples of portable ultrasound diagnosis apparatuses may include, but are not limited to, a picture archiving and communication system (PACS) viewer, a smartphone, a laptop computer, a personal digital assistant (PDA), and a tablet PC.

The probe 20 transmits ultrasound waves to an object 10 in response to a driving signal applied by the ultrasound transceiver 100 and receives echo signals reflected by the object 10. The probe 20 includes a plurality of transducers, and the plurality of transducers oscillate in response to electric signals and generate acoustic energy, that is, ultrasound waves. Furthermore, the probe 20 may be connected to the main body of the ultrasound diagnosis apparatus 1000 by wire or wirelessly, and according to embodiments, the ultrasound diagnosis apparatus 1000 may include a plurality of probes 20.

A transmitter 110 supplies a driving signal to the probe 20. The transmitter 110 includes a pulse generator 112, a transmission delaying unit 114, and a pulser 116. The pulse generator 112 generates pulses for forming transmission ultrasound waves based on a predetermined pulse repetition frequency (PRF), and the transmission delaying unit 114 delays the pulses by delay times necessary for determining transmission directionality. The pulses which have been delayed correspond to a plurality of piezoelectric vibrators included in the probe 20, respectively. The pulser 116 applies a driving signal (or a driving pulse) to the probe 20 based on timing corresponding to each of the pulses which have been delayed.

A receiver 120 generates ultrasound data by processing echo signals received from the probe 20. The receiver 120 may include an amplifier 122, an analog-to-digital converter (ADC) 124, a reception delaying unit 126, and a summing unit 128. The amplifier 122 amplifies echo signals in each channel, and the ADC 124 performs analog-to-digital conversion with respect to the amplified echo signals. The reception delaying unit 126 delays digital echo signals output by the ADC 124 by delay times necessary for determining reception directionality, and the summing unit 128 generates ultrasound data by summing the echo signals processed by the reception delaying unit 166. In some embodiments, the receiver 120 may not include the amplifier 122. In other words, if the sensitivity of the probe 20 or the capability of the ADC 124 to process bits is enhanced, the amplifier 122 may be omitted.

The image processor 200 generates an ultrasound image by scan-converting ultrasound data generated by the ultrasound transceiver 100 and displays the ultrasound image. The ultrasound image may be not only a grayscale ultrasound image obtained by scanning an object in an amplitude (A) mode, a brightness (B) mode, and a motion (M) mode, but also a Doppler image showing a movement of an object via a Doppler effect. The Doppler image may be a blood flow Doppler image showing flow of blood (also referred to as a color Doppler image), a tissue Doppler image showing a movement of tissue, or a spectral Doppler image showing a moving speed of an object as a waveform. According to exemplary embodiments, the image processor 200 may include an image demodulation apparatus.

A B mode processor 212 extracts B mode components from ultrasound data and processes the B mode components. An image generator 220 may generate an ultrasound image indicating signal intensities as brightness based on the extracted B mode components 212.

Similarly, a Doppler processor 214 may extract Doppler components from ultrasound data, and the image generator 220 may generate a Doppler image indicating a movement of an object as colors or waveforms based on the extracted Doppler components.

According to an embodiment, the image generator 220 may generate a three-dimensional (3D) ultrasound image via volume-rendering with respect to volume data and may also generate an elasticity image by imaging deformation of the object 10 due to pressure. Furthermore, the image generator 220 may display various pieces of additional information in an ultrasound image by using text and graphics. In addition, the generated ultrasound image may be stored in the memory 400.

A display 230 displays the generated ultrasound image. The display 230 may display not only an ultrasound image, but also various pieces of information processed by the ultrasound diagnosis apparatus 1000 on a screen image via a graphical user interface (GUI). In addition, the ultrasound diagnosis apparatus 1000 may include two or more displays 230 according to embodiments.

The communication module 300 is connected to a network 30 by wire or wirelessly to communicate with an external device or a server. The communication module 300 may exchange data with a hospital server or another medical apparatus in a hospital, which is connected thereto via a PACS. Furthermore, the communication module 300 may perform data communication according to the digital imaging and communications in medicine (DICOM) standard.

The communication module 300 may transmit or receive data related to diagnosis of an object, e.g., an ultrasound image, ultrasound data, and Doppler data of the object, via the network 30 and may also transmit or receive medical images captured by another medical apparatus, e.g., a computed tomography (CT) apparatus, a magnetic resonance imaging (MRI) apparatus, or an X-ray apparatus. Furthermore, the communication module 300 may receive information about a diagnosis history or medical treatment schedule of a patient from a server and utilizes the received information to diagnose the patient. Furthermore, the communication module 300 may perform data communication not only with a server or a medical apparatus in a hospital, but also with a portable terminal of a medical doctor or patient.

The communication module 300 is connected to the network 30 by wire or wirelessly to exchange data with a server 32, a medical apparatus 34, or a portable terminal 36. The communication module 300 may include one or more components for communication with external devices. For example, the communication module 1300 may include a local area communication module 310, a wired communication module 320, and a mobile communication module 330.

The local area communication module 310 refers to a module for local area communication within a predetermined distance. Examples of local area communication techniques according to an embodiment may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 320 refers to a module for communication using electric signals or optical signals. Examples of wired communication techniques according to an embodiment may include communication via a twisted pair cable, a coaxial cable, an optical fiber cable, and an Ethernet cable.

The mobile communication module 330 transmits or receives wireless signals to or from at least one selected from a base station, an external terminal, and a server on a mobile communication network. The wireless signals may be voice call signals, video call signals, or various types of data for transmission and reception of text/multimedia messages.

The memory 400 stores various data processed by the ultrasound diagnosis apparatus 1000. For example, the memory 400 may store medical data related to diagnosis of an object, such as ultrasound data and an ultrasound image that are input or output, and may also store algorithms or programs which are to be executed in the ultrasound diagnosis apparatus 1000.

The memory 400 may be any of various storage media, e.g., a flash memory, a hard disk drive, EEPROM, etc. Furthermore, the ultrasound diagnosis apparatus 1000 may utilize web storage or a cloud server that performs the storage function of the memory 400 online.

The input device 500 refers to a means via which a user inputs data for controlling the ultrasound diagnosis apparatus 1000. The input device 500 may include hardware components, such as a keypad, a mouse, a touch panel, a touch screen, and a jog switch. However, embodiments are not limited thereto, and the input device 1600 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The controller 600 may control all operations of the ultrasound diagnosis apparatus 1000. In other words, the controller 600 may control operations among the probe 20, the ultrasound transceiver 100, the image processor 200, the communication module 300, the memory 400, and the input device 500 shown in FIG. 1.

All or some of the probe 20, the ultrasound transceiver 100, the image processor 200, the communication module 300, the memory 400, the input device 500, and the controller 600 may be implemented as software modules. However, embodiments are not limited thereto, and some of the components stated above may be implemented as hardware modules. Furthermore, at least one selected from the ultrasound transceiver 100, the image processor 200, and the communication module 300 may be included in the controller 600. However, embodiments of the present invention are not limited thereto.

As described above, according to the one or more of the above exemplary embodiments, a section in which frequency estimation is valid is detected, and then, attenuation of a frequency is estimated based on the valid section, thereby providing an improved image.

The exemplary embodiment of the inventive concept may be implemented in the form of a storage medium that includes computer executable instructions, such as program modules, being executed by a computer. Non-transitory computer-readable media may be any available media that may be accessed by the computer and includes volatile media such as RAM, nonvolatile media such as ROM, and removable and non-removable media. In addition, the non-transitory computer-readable media may include computer storage media and communication media. Computer storage media includes the volatile media, non-volatile media, and removable and non-removable media implemented as any method or technology for storage of information such as computer readable instructions, data structures, program modules, or other data. The medium of communication is typically computer-readable instructions, and other data in a modulated data signal such as data structures, or program modules, or other transport mechanism and includes any information delivery media. Examples of the computer storage media include ROM, RAM, flash memory, CD, DVD, magnetic discs, or magnetic tapes.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

1. An adaptive demodulation method comprising:
acquiring input radio frequency (RF) data;
quadrature-demodulating the input RF data to output an inphase-quadrature (IQ) signal;
determining a valid region for the input RF data; and
estimating attenuation of a frequency of the IQ signal, based on data included in the valid region from among the input RF data and performing frequency compensation corresponding to the estimated attenuation of the frequency.

2. The adaptive demodulation method of claim 1, wherein step c) comprises:
acquiring a cross-correlation of the input RF data; and
determining the valid region, based on the cross-correlation.

3. The adaptive demodulation method of claim 2, wherein the acquiring of the cross-correlation comprises acquiring a cross-correlation between previous beamformed data and current beamformed data.

4. The adaptive demodulation method of claim 2, wherein, step c) further comprises performing beamforming of a virtual scan line by using ith scan line channel data and i+1st scan line channel data, and
the acquiring of the cross-correlation comprises acquiring a cross-correlation of beamformed data.

5. The adaptive demodulation method of claim 1, wherein the determining of the valid region comprises:
acquiring a signal-to-noise (SNR) value from previous beamformed data and current beamformed data; and
determining the valid region, based on the SNR value.

6. The adaptive demodulation method of claim 1, wherein step d) comprises:
acquiring an auto-correlation of the IQ signal;
performing polynomial fitting, based on the auto-correlation and the valid region; and
performing frequency shift compensation, based on a result of the polynomial fitting.

7. An adaptive demodulation apparatus comprising:
an input data acquirer that acquires input radio frequency (RF) data;
a quadrature demodulator that quadrature-demodulates the input RF data to output an inphase-quadrature (IQ) signal;
a valid region determiner that determines a valid region for the input RF data; and
a frequency compensator that estimates attenuation of a frequency of the IQ signal, based on data included in the valid region from among the input RF data and performs frequency compensation corresponding to the estimated attenuation of the frequency.

8. The adaptive demodulation apparatus of claim 7, wherein the valid region determiner comprises:
a cross-correlator that acquires a cross-correlation of the input RF data;
a polynomial function fitting unit that performs polynomial fitting on the input RF data, based on the cross-correlation; and
a valid region selector that determines the valid region, based on a result of the polynomial fitting.

9. The adaptive demodulation apparatus of claim 8, wherein the cross-correlator acquires a cross-correlation between previous beamformed data and current beamformed data.

10. The adaptive demodulation apparatus of claim 7, wherein the valid region determiner comprises:
a beamformer that performs beamforming of a virtual scan line by using ith scan line channel data and i+1st scan line channel data;
a cross-correlator that acquires a cross-correlation, based on the virtual scan line;
a polynomial function fitting unit that performs polynomial fitting, based on the cross-correlation; and
a valid region selector that determines the valid region, based on a result of the polynomial fitting by the polynomial function fitting unit.

11. The adaptive demodulation apparatus of claim 7, wherein the valid region determiner comprises:
a signal-to-noise (SNR) estimator that estimates an SNR value from previous beamformed data and current beamformed data;
a polynomial function fitting unit that performs polynomial fitting, based on the estimated SNR value; and
a valid region selector that determines the valid region, based on a result of the polynomial fitting by the polynomial function fitting unit.

12. The adaptive demodulation apparatus of claim 7, wherein the frequency compensator comprises:
an auto-correlator that acquires an auto-correlation of the IQ signal;
a polynomial function fitting unit that performs polynomial fitting, based on the auto-correlation and the valid region; and
a frequency shift compensator that performs frequency shift compensation, based on a result of the polynomial fitting.

13. A non-transitory computer-readable storage medium storing a program for executing the adaptive demodulation method of claim 1.
